(19) United States
(12) Patent Application Publication
(10) Pub. No.: US 2012/0282236 A1
(43) Pub. Date: Nov. 8, 2012

(54) METHOD OF DELIVERING OXYGEN USING PEG-HEMOGLOBIN CONJUGATES WITH ENHANCED NITRITE REDUCTASE ACTIVITY

(75) Inventors: Kim D. Vandegriff, San Diego, CA (US); Ashok Malavalli, San Diego, CA (US); Scott D. Olsen, San Diego, CA (US)

(73) Assignee: SANGART, INC., San Diego, CA (US)

(21) Appl. No.: 13/461,577

(22) Filed: May 1, 2012

Related U.S. Application Data
(60) Provisional application No. 61/481,684, filed on May 2, 2011.

(51) Int. Cl. A61K 38/44; A61P 7/00

(52) U.S. Cl. 424/94.3

(57) ABSTRACT

The present invention relates generally to methods for delivering oxygen to tissue and reducing nitrite to nitric oxide in the microvasculature. Specifically, the present invention is directed towards using a deoxygenated pegylated hemoglobin conjugate having enhanced nitrite reductase activity to deliver oxygen to tissues.

### CROSS-REFERENCE TO RELATED APPLICATIONS

This application is a continuation of U.S. Provisional Patent Application No. 61/481,684 entitled “Method of Delivering Oxygen using PEG-Hemoglobin Conjugates with Enhanced Nitrite Reductase Activity” and filed May 2, 2011, the contents of which are incorporated herein in their entirety.

### TECHNICAL FIELD

The present invention relates generally to methods for delivering oxygen to tissue and reducing nitrite to nitric oxide in the microvasculature. Specifically, the present invention is directed towards using a deoxygenated pegylated hemoglobin conjugate having enhanced nitrite reductase activity to deliver oxygen to tissues.

### BACKGROUND OF THE INVENTION

Hemoglobin-based oxygen carriers (“HBOC”) have long been associated with vasoconstriction that has been attributed to nitric oxide (NO) scavenging by heme. Oxygen carriers that are useful as oxygen therapeutics (sometimes referred to as “oxygen-carrying plasma expanders”), such as stabilized hemoglobin (Hb), have been shown to have limited efficacy because they scavenge nitric oxide, causing vasoconstriction and hypertension. While the specific cause has not as yet been determined, one school of thought suggests the possibility that the heme iron may combine rapidly and irreversibly with endogenous NO, thereby causing vasoconstriction. Thus, no oxygen carrier to date has been entirely successful as an oxygen therapeutic, though products comprising modified cell-free Hb are thought to be the most promising.

As alluded to, some of the physiological effects of these oxygen carrying solutions are not fully understood. Of these, perhaps the most controversial is the propensity to cause vasoconstriction, which may manifest as hypertension in animals and man (Amberson, W., 1947, Science 106:117-1)7) (Keipert, P. et al., 1993, Transfusion 33:701-708). Human Hb cross-linked linked between α-chains with bis-dibromosalicyl-fumarate (“ααHb”) was developed by the U.S. Army as a model red cell substitute, but was abandoned after it showed severe increases in pulmonary and systemic vascular resistance (Hess, J. et al., 199), Blood 78:356A). A commercial version of this product was also abandoned after a disappointing Phase III clinical trial (Winslow, R. M., 2000, Vox Sang 79:1-20).

The most common explanation for the vasoconstriction produced by cell-free Hb is that it readily binds the endothelium-derived relaxing factor (EDRF), nitric oxide (“NO”). Two molecular approaches have been advanced in attempting to overcome the NO binding activity of Hb. The first approach was utilizing recombinant DNA, which attempted to reduce the NO binding of Hb by site-specific mutagenesis of the distal heme pocket (Eich, R. F. et al., 1996, Biochem. 35:6976-83). The second approach utilized chemical modification in which the size of the Hb was enhanced through oligomerization, which attempted to reduce or possibly completely inhibit the extravasation of Hb from the vascular space into the interstitial space (Hess, J. R. et al., 1978, J. Appl. Physiol. 74:1769-78; Muldoon, S. M. et al., 1996, J. Lab. Clin. Med. 128:579-83; Macdonal, V. W. et al., 1994, Biotechnology 22:565-75; Furchgott, R., 1984, Ann. Rev. Pharmacol. 24:175-97; and Kilbourne, R. et al., 1994, Biochem. Biophys. Res. Commun. 199:155-62).

In fact, recombinant Hbs with reduced affinity for NO have been produced that are less hypertensive in top-load rat experiments (Doherty, D. H. etg al. 1998, Nature Biotechnology 16:672-676 and Lemon, D. D. et al. 1996, Biotech 24:378). However, studies suggest that NO binding may not be the only explanation for the vasoactivity of Hb. It has been found that certain large Hb molecules, such as those modified with PEG, were virtually free of the hypertensive effect, even though their NO binding rates were identical to those of the severely hypertensive ααHb (Rohlfs, R. J. et al. 1998, J Biol. Chem. 273:12128-12)34). Furthermore, it was found that PEG-Hb was extraordinarily effective in preventing the consequences of hemorrhage when given as an exchange transfusion prior to hemorrhage (Winslow, R. M. et al. 1998, J. Appl. Physiol. 85:993-1003).

The conjugation of PEG to Hb reduces its antigenicity and extends its circulation half-life. However, the PEG conjugation reaction has been reported to result in dissociation of Hb tetramers into αβ-dimer subunits causing gross hemoglobinuria in exchange-transfused rats receiving PEG-conjugates of Hb monomeric units below 40,000 Daltons (“Da”) (Iwashita and Ajisaka Organ-Directed Toxicity: Chem. Indicies Mech., Proc. Symp., Brown et al. 1981, Eds. Pergamon, Oxford, England pgs 97-101). A polyalkylene oxide (“PAO”) conjugated Hb having a molecular weight greater than 84,000 Da was prepared by Enzon, Inc. (U.S. Pat. No. 5,650,388) that carried 10 copies of PEG-5,000 chains linked to Hb at its α and ε-amino groups. This degree of substitution was described as avoiding clinically significant nephrotoxicity associated with hemoglobinuria in mammals. However, the conjugation reaction resulted in a heterogeneous conjugate population and contained other undesirable reactants that had to be removed by column chromatography.

PEG conjugation is typically carried out through the reaction of an activated PEG with a functional group on the surface of biomolecules. The most common functional groups are the amino groups of lysine and histidine residues, and the N-terminus of proteins; thiol groups of cysteine residues; and the hydroxyl groups of serine, threonine and tyrosine residues and the C-terminus of the protein. PEG is usually activated by converting the hydroxyl terminus to a reactive moiety capable of reacting with these functional groups in a mild aqueous environment. One of the most common monofunctional PEGs used for conjugation of therapeutic biopharmaceuticals is methoxy-PEG (“mPEG”), which has only one functional group (i.e. hydroxyl), thus minimizing cross-linking and aggregation problems that are associated with bifunctional PEG. However, mPEG is often contaminated with high molecular weight bifunctional PEG (i.e. “PEG diol”), which can range as high as 10 to 15% (Dust J. M. et al. 1990, Macromolecule 23:3742-3746), due to its production process. This bifunctional PEG diol has roughly twice the size of the desired monofunctional PEG. The contamination problem is further aggravated as the molecular weight of PEG increases. The purity of mPEG is especially critical for the production of PEGylated biotherapeutics, because the FDA requires a high level of reproducibility in the production processes and quality of the final drug product.

Conjugation of Hb to PAOs has been performed in both the oxygenated and deoxygenated states. U.S. Pat. No. 6,844,317 describes conjugating Hb in the oxygenated, or “R” state, to enhance the oxygen affinity of the resultant PEG-Hb conjugate. This is accomplished by equilibrating Hb with the atmosphere prior to conjugation. Others describe a deoxygenation step prior to conjugation to diminish the oxygen affinity and increase structural stability enabling the Hb to withstand the physical stresses of chemical modification, diafiltration and/or sterile filtration and sterilization (U.S. Pat. No. 5,234,903). For intramolecular cross-linking of Hb, it is suggested that deoxygenating Hb prior to modification may be required to expose lysine 99, of the α-chain, to the cross-linking reagent (U.S. Pat. No. 5,234,903).

The kinetics of Hb thiolation with iminothiolane prior to conjugation with PEG was investigated by Acharya et al. (U.S. Pat. No. 7,501,499). It was observed that increasing the concentration of iminothiolane from 10-fold, which introduced an average of five extrinsic thiols per tetramer, to 30-fold nearly doubled the number of extrinsic thiols on Hb. However, the size enhancement seen after PEG conjugation was only marginal, even with double the number of thiols. This suggested that the conjugation reaction in the presence of 20-fold molar excess of maleimidyl PEG-5000 covered the surface of the Hb with less reactive thiols resulting in steric interference that resisted further modification of Hb with more reactive thiols. Consequently, to achieve the desired molecular weight of modified Hb (i.e. 6±1 PEG per Hb molecule), Acharya et thiolated Hb with an 8-15 molar excess of iminothiolane, and then reacted the thiolated Hb with a 16-30 fold molar excess of maleimidyl PEG-5000. However, these high molar excess reactant concentrations in large scale production significantly increase the cost for preparing the HBOC. Moreover, such high molar excess of the maleimidyl PEG-5000 results in a more heterogeneous product with the production of a greater number of unwanted reactants.

Recently, evidence has been presented that reduction of nitrite to NO by deoxyhemoglobin has the ability to vasodilate blood vessels (Cosby, K. et al. 2003, Nat. Med. 9:1498). It is believed that this nitrite reductase activity of hemoglobin is under allosteric control and produces NO at a maximal rate when deoxyhemes are in an R-state conformation. Further, it has been shown that while cell-free Hbs caused vasoconstriction and reduced perfusion, PEG-Hbs maintained blood flow and microvascular perfusion pressure, which is thought to be related to the lack of vasoconstriction (Tsai, A. G. et al. 2006, Blood 108:3603). Other studies also suggest that the modification of cell-free hemoglobin derivatives with multiple chains of PEG may suppress vasoactivity. Experiments utilizing R-State stabilized Hbs with five to six PEG chains demonstrated 10-fold faster nitrite reductase activity as compared to native Hb (Lui, F. E. et al. 2008, Biochemistry 47(40), 10773-10780). However, it was concluded that any further PEG conjugation at accessible lysine residues did not contribute to increased nitrite reductase activity.

Consequently, there is a need for a method of delivering oxygen to tissue and reducing nitrite to nitric oxide in the microvasculature through the use of a deoxygenated PEG-Hb having increased nitrite reductase properties compared to native or stroma free Hb.

### SUMMARY OF THE INVENTION

The present invention relates generally to methods of delivering oxygen to tissue and reducing nitrite to nitric oxide in the microvasculature. Specifically, the present invention is directed towards using a deoxygenated pegylated hemoglobin conjugate having enhanced nitrite reductase activity to deliver oxygen to tissues.

Exemplary embodiments of the invention relate to a method for delivering oxygen to tissue and reducing nitrite to nitric oxide (NO) in the microvasculature comprising administering a deoxygenated maleimide polyethylene glycol hemoglobin (MalPEG-Hb) conjugate to a subject, wherein the deoxygenated MalPEG-Hb conjugate has at least 20-fold greater nitrite reductase activity compared to that of stroma free hemoglobin when measured under the same conditions.

Other aspects of the invention are found throughout the specification.

### DETAILED DESCRIPTION OF THE INVENTION

The present invention relates generally to methods for delivering oxygen to tissue and reducing nitrite to nitric oxide in the microvasculature. Specifically, the present invention is directed towards using a deoxygenated pegylated hemoglobin conjugate having enhanced nitrite reductase activity to deliver oxygen to tissues.

In the description that follows, a number of terms used in the field of hemoglobin research and medicine are extensively utilized. In order to provide a clear and consistent understanding of the specification and claims, including the scope to be given such terms, the following non-limiting definitions are provided.

When the terms “one,” “a” or “an” are used in this disclosure, they mean “at least one” or “one or more,” unless otherwise indicated.

The terms “activated polyalkylene oxide” or “activated PAO” as used herein refer to a PAO molecule that has at least one functional group. A functional group is a reactive moiety that interacts with free amines, sulfhydryls or carboxyl groups on a molecule to be conjugated with PAO. For example, one such functional group that reacts with free sulfhydryls is a maleimide group. Correspondingly, a functional group that reacts with free amines is a succinimide group.

The terms “hemoglobin” or “Hb” as used herein refer generally to the protein within red blood cells that transports oxygen. Each molecule of Hb has 4 subunits, 2 α-chain subunits and 2 β-chain subunits, which are arranged in a tetrameric structure. Each subunit also contains one heme group, which is the iron-containing center that binds the ligands O₂, NO and CO. Thus, each Hb molecule can bind up to 4 ligand molecules.

The term “MalPEG-Hb” as used herein refers to Hb to which maleimidyl-activated PEG has been conjugated. The conjugation is performed by reacting MalPEG with surface thiol groups (and to a lesser extent, amino groups) on the Hb to form MalPEG-Hb. Thiol groups are found in cysteine residues present in the amino acid sequence of Hb, and can also be introduced by modifying surface amino groups to contain a thiol group.

The terms “methemoglobin” or “metHb” as used herein refer to an oxidized form of Hb that contains iron in the ferric state. MetHb does not function as a ligand carrier. The term “methemoglobin %” as used herein refers to the percentage of oxidized Hb to total Hb.

The terms “methoxy-PEG” or “mPEG” as used herein refer to PEG wherein the hydrogen of the hydroxyl terminus is replaced with a methyl (—CH₃) group.

The terms “mixture” or “mixing” as used herein refer to a mingling together of two or more substances without the occurrence of a reaction by which they would lose their individual properties.

The term “solution” refers to a liquid mixture and the term “aqueous solution” refers to a solution that contains some water and may also contain one or more other liquid substances with water to form a multi-component solution.

The terms “modified hemoglobin” or “modified Hb” as used herein refer to, but are not limited to. Hb that has been altered by a chemical reaction, such as intra- and inter-molecular crosslinking, and recombinant techniques, such that the Hb is no longer in its “native” state. As used herein, the terms “hemoglobin” or “Hb” refer to both native unmodified Hb and modified Hb, unless otherwise indicated.

The term “oxygen affinity” as used herein refers to the avidity with which an oxygen carrier, such as Hb, binds molecular oxygen. This characteristic is defined by the oxygen equilibrium curve, which relates the degree of saturation of Hb molecules with oxygen (Y axis) with the partial pressure of oxygen (X axis). The position of this curve is denoted by the P50 value, which is the partial pressure of oxygen at which the oxygen carrier is half-saturated with oxygen, and is inversely related to oxygen affinity. Hence, the lower the P50, the higher the oxygen affinity. The oxygen affinity of whole blood (and components of whole blood, such as red blood cells and Hb) can be measured by a variety of methods known in the art. (see, e.g., Winslow, R. M. et al., J. Biol. Chem.)977, 252:2331-37). Oxygen affinity may also be determined using a commercially available HEMOX™ Analyzer (TCS Scientific Corporation, New Hope, Pa.). (see, e.g., Vandegriff and Shrager in “Methods in Enzymology” (Everse et al., eds.) 232:460 (1994)).

The terms “polyethylene glycol” or “PEG” as used herein refer to polymers of the general chemical formula H(OCH₂CH₂)ₙOH, also known as (α-Hydro-ω-hydroxypoly-(oxy-1,2-ethanediyl), where “n” is greater than or equal to 4. Any PEG formulation, substituted or unsubstituted, is encompassed by this term. PEGs are commercially available in a number of formulations (e.g., Carbowax™ (Dow Chemical, Midland, Mich.) and Poly-G® (Arch Chemicals, Norwalk, Conn.)).

The terms “polyethylene glycol-conjugated hemoglobin,” “PEG-Hb conjugate” or “PEG-Hb” as used herein refer to Hb to which PEG is covalently attached.

The terms “stroma-free hemoglobin” or “SFH” as used herein refer to Hb from which all red blood cell membranes have been removed.

The term “surface-modified hemoglobin” as used herein refers to hemoglobin to which chemical groups, usually polymers, have been attached, such as dextran or polyalkylene oxide. The term “surface modified oxygenated hemoglobin” refers to Hb that is in the “R” state when it is surface modified.

The term “thiolation” as used herein refers to a process that increases the number of sulfhydryl groups on a molecule. For example, reacting a protein with 2-iminothiolane (“2-IT”) converts free amines on the surface of a protein to sulfhydryl groups.

### Hemoglobin-Based Oxygen Carriers

A variety of PAO-Hbs that have or demonstrate an oxygen affinity greater than whole blood may be utilized with the present invention. This means that the PAO-Hbs will have a p50 greater than 3, but less than 10 mmHg. These p50 values translate into a higher O₂ binding affinity than SFH, which has a p50 of approximately 15 mmHg, and a significantly higher O₂ binding affinity than whole blood, which has a p50 of approximately 28 mmHg.

The idea that increasing oxygen affinity of an HBOC over that of whole blood as a method to enhance oxygen delivery to tissues contradicts the widely held belief that modified Hb blood substitutes should have lower oxygen affinities. The previous belief held that HBOCs should have p50s that approximated that of whole blood to effectively release oxygen to tissue. Because of this, many researchers modified Hb with pyridoxyl phosphate to raise the p50 of SFH from 10 mmHg to approximately 22 mmHg.

1. Organic Polymers

In previous studies, it was observed that the molecular size of surface modified hemoglobin has to be large enough to avoid being cleared by the kidneys and to achieve the desired circulation half-life. Blumenstein, J. et al., determined that this could be achieved at, or above, a molecular weight of 84,000 Daltons (“Da”) (“Blood Substitutes and Plasma Expanders,” Alan R. Liss, editors, New York, N.Y., pages 205-212 (1978)). In that study, the authors conjugated dextran of varying molecular weight to Hb. They reported that a conjugate of Hb (with a molecular weight of 64,000 Da) and dextran (having a molecular weight of 20,000 Da) “was cleared slowly from the circulation and negligibly through the kidneys.” Further, it was observed that increasing the molecular weight above 84,000 Da did not significantly alter these clearance curves.

The present invention may be utilized with a variety of PAO-Hb conjugates having a molecular weight of at least 84,000 Da. Suitable PAO polymers used in preparing these conjugates include for example, polyethylene oxide (—(CH₂CH₂O)ₙ—), polypropylene oxide (—(CH(CH₃)CH₂O)ₙ—) and a polyethylene/polypropylene oxide copolymer (—(CH₂CH₂O)ₙ—(CH(CH₃)CH₂O)ₙ—). Other straight or branched chain and optionally substituted synthetic polymers that would be suitable in the practice of the present invention are well known in the medical field.

The most common PAO presently used to modify the surface of Hb is PEG because of its pharmaceutical acceptability and commercial availability. In addition, PEG is available in a variety of molecular weights based on the number of repeating subunits of ethylene oxide (i.e. —OCH₂CH₂—) within the molecule. Consequently, PEG also provides the flexibility of achieving a desired molecular weight based on the number and size of the PEG molecules conjugated to Hb.

In order to conjugate PAO to Hb, one or both of the terminal end groups of the PAO polymer must first be converted into a reactive functional group. This process is referred to as “activation.” In one well known process, PEG-OH is used to prepare PEG-halide, mesylate or tosylate, which is then converted to PEG-amine (“PEG-NH₂”) by performing a nucleophilic displacement reaction. The displacement reaction can be performed with aqueous ammonia (Zalipsky, S. et al., 1983, Eur. Polym. J. 19:1177-1183), sodium azide or potassium phthalimide. The activated PEG can then be conjugated to a biological molecule through the interaction of the PEG amine group (—“NH₂”) with a carboxyl group (“—COOH”) of the biological molecule.

PEG-NH, can be further functionalized to conjugate with groups other than —COOH. For example, U.S. Pat. No. 6,828,401 discloses the reaction of PEG-NH, with maleimide to form mPEG-maleimide. In this reaction, mPEG-OH is reacted with a tosylating reagent (p-toluenesulfonyl chloride) and a base catalyst (triethyleneamine) in the presence of an organic solvent (dichloromethane) to produce mPEG-tosylate. The mPEG-tosylate is then reacted with 28% ammonia water and maleic acid anhydride in an organic solvent mixture of N,N-dimethylacetamide (“DMAc”) and N-cyclohexylpyrrolidinone (“CHP”) to produce a maleamic acid compound. This compound is then reacted with pentafluorophenyl trifluoroacetate in the presence of dichloromethane to produce the mPEG-maleimide.

In addition, linkers have been used to conjugate PAO to Hb. These linkers do not generally affect the performance of the surface modified Hb. However, rigid linkers are preferred over flexible linkers because they enhance the manufacturing and/or characteristics of the conjugates. Desired rigid linkers include unsaturated aliphatic or aromatic C₁ to C₆ linker substituents.

2. Hemoglobin

A variety of Hbs may be utilized with the present invention. The Hb may be obtained from animal sources or produced by recombinant techniques. Human Hb is desirable in the present invention and can be obtained from natural sources. Further, the genes of both human α- and β-globin have been both cloned and sequenced (Liebhaber, S. A. et al., 1980, PNAS 77:7054-7058 and Marotta, C. A. et al., 1977, J. Biol. Chem. 353: 5040-5053). Consequently, human Hb can also be recombinantly engineered. In addition, many recombinantly modified Hbs have been produced using site-directed mutagenesis. Unfortunately, these “mutant” Hb varieties have undesirably high oxygen affinities (e.g., Nagai, K. et al., 1985, PNAS 82:7252-7255).

Native human Hb has a fixed number of amino acid residue side chains that may be accessed for conjugation to maleimide-activated PAO molecules. These are presented in the chart below:

| | |
|---|---|
| Residues | Positions |

α-chain

| | |
|---|---|
| Lys | 7, 11, 16, 40, 56, 60, 61, |
| | 90, 99, 127 and 139 |
| Cys | 104 |
| His | 20, 45, 50, 58, 72, 87, |
| | 112 and 122 |
| Val | 1 |

β-chain

| | |
|---|---|
| Lys | 8, 17, 59, 61, 65, 66, 82, |
| | 95, 120, 132 and 144 |
| Cys | 93 and 112 |
| His | 2, 63, 77, 92, 97, 116, |
| | 117, 143 and 146 |
| Val | 1 |

One method to increase the number of available conjugation sites on Hb is to introduce sulfhydryl groups (also known as thiolation), which tend to be more reactive with PEG-Mal than free amines. A variety of methods are known for protein thiolation. In one method, protein free amines are reacted with succinimidyl 3-(2-pyridyldithio) propionate followed by reduction with dithiothreitol (“DTT”), or tris(2-carboxyethyl)phosphine (“TCEP”). This reaction releases the 2-pyridinethione chromophore, which can be used to determine the degree of thiolation. Amines can also be indirectly thiolated by reaction with succinimidyl acetylthioacetate, followed by 50 mM hydroxylamine, or hydrazine at near-neutral pH.

Another method described in U.S. Pat. No. 5,585,484 maintains the positive charge of the amino (α- or ε-) group of the Hb after conjugation. This method involves amidination of the ε-amino groups of Hb by 2-IT to introduce sulfhydryl groups onto the protein. This approach has at least two additional advantages over the previously used succinimidyl chemistry: 1) the high reactivity and selectivity of maleimide groups with sulfhydryl groups facilitates the near quantitative modification of the thiols, with a limited excess of reagents and 2) the thiol group of 2-IT is latent and is generated only in situ as a consequence of the reaction of the reagent with the protein amino groups. These advantages provide one additional benefit. They allow simultaneous incubation of Hb with both the thiolating and PEGylation reagent for surface decoration.

3. Conjugation

The molecular weight of the PAO-Hb may be regulated by the conjugation reaction. Conventional thought suggested that increasing the molar ratios of the reactants would increase the number of PEG molecules bound to Hb. This included both the thiolation process of Hb (i.e. increasing the molar ratio of thiolating agent to Hb) and the conjugation process (i.e. increasing the molar ratio of thiol activated PEG to thiolated Hb). However, these excess molar ratios resulted in the binding of only 6±1 PEG molecules per Hb (see U.S. Pat. No. 7,501,499).

Recently it was determined that a greater number of PAO molecules could be bound to Hb using lower molar ratios of reactants. The number of available thiol groups on Hb, before and after thiolation and after conjugation, was determined using the dithiopyridine colorimetric assay (Ampulski, R. S. et al., 1969, Biochem. Biophys. Acta 32:163-169). Human Hb contains two intrinsic reactive thiol groups at the β93cysteine residues, which was confirmed by the dithiopyridine reaction. After thiolation of SFH with 2-IT, the number of reactive thiol groups increased from two to over seven. In this example, an average of 8 PEG molecules was bound to Hb. This was achieved using a 7.5 molar excess of 2-IT over SFH in the thiolation reaction and a 12 molar excess of PEG-Mal over thiolated Hb in the conjugation reaction.

4. PEG-Hb Conjugate

The PEG-Hb conjugate of the present invention has an oxygen affinity greater than whole blood. This means that the conjugate will have a p50 greater than 3, but less than 10 mmHg. These p50 values translate into a higher O₂ binding affinity than SFH, which has a p50 of approximately 15 mmHg and a significantly higher O₂ binding affinity than whole blood, which has a p50 of approximately 28 mmHg. It was suggested that increasing oxygen affinity of HBOC, and thereby lowering the p50, could enhance delivery of oxygen to tissues, but that a p50 lower than that of SFH would not be acceptable. See Winslow, R. M. et al., in “Advances in Blood Substitutes” (1997), Birkäuser, eds. Boston, Mass., at page 167, and U.S. Pat. No. 6,054,427. This suggestion contradicts the widely held belief that HBOCs should have lower oxygen affinities similar to that of whole blood. Consequently, many researchers have used pyridoxyl phosphate to raise the p50 of SFH from 10 mmHg to approximately 22 mmHg.

There are a number of scientific approaches to manufacturing HBOCs with high oxygen affinity. Recent studies have identified the β93 cysteine residue as playing an important role in oxygen affinity. The β92 histidine residue, which is the only residue in the β-subunit directly coordinated to the heme iron, is located immediately adjacent the β93 cysteine residue. This β93 cysteine residue forms a salt bridge with the heme that normally stabilizes the low-affinity T-state Hb conformation (Perutz, M. F. et al., 1974, Biochemistry 13:2163-2173). However, attachment of the bulky maleimide group of PEG-Mal to the β93 cysteine displaces this salt bridge and shifts the quaternary conformation towards the R state, resulting in higher O₂ affinity (Imai, K. et al., 1973, Biochemistry, 12:798-807). Because of these findings, site-directed mutagenesis has now been performed to manipulate oxygen affinity to the desired level (see, e.g., U.S. Pat. No. 5,661,124). Other approaches are discussed in U.S. Pat. No. 6,054,427.

In previous studies, it was observed that the molecular size of the resultant modified Hb had to be large enough to avoid being cleared by the kidneys and to achieve the desired circulation half-life. Blumenstein, J. et al. (supra), determined that this could be achieved at or above a molecular weight of 84,000 Da. Because of this, the Hb of a number of HBOCs is crosslinked; meaning that the tetrameric hemoglobin units have been chemically bound or intramolecularly crosslinked to prevent dissociation into dimers. A variety of methods are known in the art for intramolecularly crosslinking Hb. Chemical crosslinking reagents include glutaraldehyde (U.S. Pat. No. 7,005,414), polyaldehydes (U.S. Pat. No. 4,857,636), diaspirin (U.S. Pat. No. 4,529,719), pyridoxyl 5′-phosphate (U.S. Pat. No. 4,529,719) and trimesoyl tris(methyl phosphate) (U.S. Pat. No. 5,250,665). Hbs also may be polymerized by intermolecular crosslinking. U.S. Pat. No. 5,895,810 describes obtaining Hb polymers of up to twelve tetramers using the same or multiple crosslinking reagents. Mixtures containing two or more different species of intermolecularly and intramolecularly crosslinked hemoglobin also have been disclosed. Unlike previous methods, the present invention does not crosslink Hb to achieve a desired molecular weight. In contrast, Hbs are conjugated to PAOs to increase their molecular weight.

4. Deoxygenation

Deoxygenation of HBOCs may be performed by any method known in the art. One simple method is to expose the HBOC solution to an inert gas, such as nitrogen, argon or helium. To assure that deoxygenation is relatively homogeneous, the HBOC solution is circulated in this process. Monitoring deoxygenation to attain desired levels may be performed by using a Co-oximeter 682 (Instrument Laboratories). If partial reoxygenation is desired, deoxygenated Hb may be exposed to oxygen or to gas mixture containing oxygen.

Alternatively, gas exchange may be accomplished through a gas-permeable membrane, such as a polypropylene or cellulose acetate membrane. Commercially available gas-exchange devices utilizing these membranes include the Celgard™ polypropylene microporous hollow fiber device from Hoechst-Celanese (Dallas, Tex.) or the Cell-Pharm™ hollow fiber oxygenator from American Laboratory (East Lyme, Conn.). In the Hoechst-Celanese Celgard™ device, oxygenated Hb is deoxygenated by passing an aqueous Hb solution through polypropylene microporous hollow filters at 10-100 ml/min/ft² while the system is purged with nitrogen at 5-20 psi. The Hb is generally circulated for about 5 to 30 minutes to achieve the desired percentage of deoxyHb. Another method for producing deoxygenated Hb comprises exposing a Hb solution to a chemical reducing agent such as, sodium ascorbate, sodium dithionate and sodium bisulfite. Hb is partially deoxygenated by adjusting the reducing agent concentration, reaction time and temperature. Alternatively, a reducing agent may be used to substantially deoxygenate Hb, and then oxygen may be reintroduced to form a partially deoxygenated product. In one embodiment of the invention, Hb is exposed to a 100 mM concentration of sodium bisulfite for about one hour prior to the addition of antioxidants.

### Nitrite Reductase Activity

Nitrite reacts with oxy- and deoxy-hemoglobin to form methemoglobin and methemoglobin+nitric oxide, respectively. The vasodilatory effect of nitrite differs from that of traditional NO donors in the presence of hemoglobin and can in part be explained by the nitrite reductase activity of hemoglobin. See Crawford et al. 2006 Blood 107:566-574; Huang et al. 2005 J Biol Chem 280:31126-31131; Huang et al. 2005 J Clin Invest 115:2099-2107. Further, generation of NO from nitrite and hemoglobin generally requires both hypoxia and an acidic environment which are present in hypoxic tissues. This allows for maximal NO generation by the deoxyheme-nitrite allosteric reaction as hemoglobin deoxygenates within the circulation.

Studies have shown that nitrite is converted to NO only through reaction with deoxyhemoglobin, and further, that faster reduction of nitrite occurs where the protein is in the relaxed or R-state conformation. Additionally, the R-state stabilizing effect that results from modification of the protein side chains may not be the sole cause of increased nitrite reductase activity, as modifications at βCys93 sites such as PEG conjugation also results in increased nitrite reductase activity. As such, it was discovered that PEG-Hb conjugates prepared according to the present invention possess unexpectedly higher nitrite reductase activity following deoxygenation. It is believed that because PEG-Hb conjugates of the present invention possess unexpectedly higher nitrite reductase activity because the methods described herein produce a PEG-Hb conjugate that is stabilized in the R-state conformation due to PEG conjugation at the βCys93 sites. Thus, where R-state conformation and βCys93 modification may contribute separately to increased nitrite reductase activity, PEG-Hb conjugates prepared according to the methods of the present invention demonstrate an even more pronounced nitrite reductase activity, thereby leading to greater therapeutic vasodilatory effects compared to stroma free Hb alone or other oxygen carriers.

### Formulation for In Vivo Administration

The PEG-Hb conjugate of the present invention is formulated in an aqueous diluents that is suitable for in vivo administration. Although the concentration of the oxygen carrier in the diluent may vary according to the application, it does not usually exceed a concentration of 10 g/dl of Hb, because of the enhanced oxygen delivery and therapeutic effects of the PEG-Hb conjugate. More specifically, the concentration is usually between 0.1 and 8 g/dl Hb.

Suitable aqueous diluents (i.e., those that are pharmaceutically acceptable for intravenous injection) include, inter alia, aqueous solutions of proteins, glycoproteins, polysaccharides, and other colloids. It is not intended that these embodiments be limited to any particular diluent. Consequently, diluents may encompass aqueous cell-free solutions of albumin, other colloids, or other non-oxygen carrying components.

This solution property of a PEG-Hb conjugate is due to the strong interaction between PEG chains and solvent water molecules. This is believed to be an important attribute for an HBOC for two reasons: 1) higher viscosity decreases the diffusion constant of both the PEG-Hb molecule, and 2) higher viscosity increases the shear stress of the solution flowing against the endothelial wall, eliciting the release of vasodilators to counteract vasoconstriction. Accordingly, the formulation of PEG-Hb in the aqueous diluent usually has a viscosity of at least 2 centipoise (cP). More specifically, between 2 and 4 cP, and particularly around 2.5 cP. In other embodiments, the viscosity of the aqueous solution may be 6 cP or greater, but is usually not more than 8 cP.

The PEG-Hb conjugate is suitable for use as a hemoglobin-based oxygen carrier as is any other such product. For example, it is useful as a blood substitute, for organ preservation, to promote hemodynamic stability during surgery, etc.

### EXAMPLES

### Example 1

### Thiolation of Hb

### A. Production of SFH

Packed red blood cells (“RBCs”) are procured from a commercial source, such as from a local Blood Bank, the New York Blood Center, or the American Red Cross. The material is obtained not more than 45 days from the time of collection. All units are screened for viral infection and subjected to nucleic acid testing prior to use. Non-leukodepleted pooled units are leukodepleted by membrane filtration to remove white blood cells. Packed RBCs are pooled into a sterile vessel and stored at 2-15° C. until further processing. The volume is noted, and Hb concentration is determined using a commercially available co-oximeter, or other art-recognized method.

RBCs are washed with six volumes of 0.9% sodium chloride using a 0.45-μm tangential flow filtration, followed by cell lysis by decreasing the concentration of salt. Hb extraction is performed using the same membrane. The cell wash is analyzed to verify removal of plasma components by a spectrophotometric assay for albumin. The lysate is processed through a 0.16-μm membrane in the cold to purify Hb. The purified Hb is collected in a sterile depyrogenated and then ultrafiltered to remove virus. Additional viral-reduction steps, including solvent/detergent treatment, nanofiltration, and anion Q membrane purification may be performed. All steps in this process are carried out at 2-15° C.

Hb from lysate is exchanged into Ringer's lactate (“RL”), or phosphate-buffered saline (“PBS”, pH 7.4), using a 30-kD membrane. The Hb is concentrated to 1.1-1.5 mM (in tetramer). Ten to 12 volumes of RL or PBS are used for solvent exchange. This process is carried out at 2-15° C. The pH of the solution prepared in RL or PBS is adjusted to 8.0 prior to thiolation. The Hb is sterile-filtered through a 0.45 or 0.2-μm disposable filter capsule and stored at 4±2° C. before the chemical modification reaction is performed.

### B. Thiolation of the SFH

Using the SFH prepared as described above, thiolation is carried out using less than 8-fold molar excess of 2-IT over Hb. The ratio and reaction time are optimized to maximize the number of thiol groups for PEG conjugation and to minimize product heterogeneity. Approximately 1 mM Hb (tetramer) in RL (pH 7.0-8.5), PBS or any similar buffer, is combined with less than 8 mM 2-IT in the same buffer. This mixture is continuously stirred for less than 6 hours at 10±5° C.

The dithiopyridine colorimetric assay (Ampulski, R. S. et al., Biochem. Biophys. Acta 1969, 32:163-169) is used to measure the number of available thiol groups on the surface of the Hb tetramer before and after thiolation, and then again after Hb-PEG conjugation. Human Hb contains two intrinsic reactive thiol groups at the β93cysteine residues, which is confirmed by the dithiopyridine reaction. After thiolation of SFH at a ratio of 1:<8 (SFH: 2-IT), the number of reactive thiol groups increases from two to greater than seven thiols.

### Example 2

### Conjugation of Hb to PEG-Mal

PEG-Mal is conjugated to the thiolated Hb from Example 1 using less than a 15-fold molar excess of PEG-Mal based on 100% terminal activity over the starting tetrameric Hb concentration. The Hb is first allowed to equilibrate with the atmosphere to oxygenate the Hb. Approximately, 1 mM thiolated Hb in RL (pH 7.0-8.5), PBS or any similar buffer is combined with less than 15 mM PEG-Mal in the same buffer. This mixture is continuously stirred for less than 6 hours at 10±5° C.

PEG-Hb conjugate is processed through a 70-kD membrane (i.e. <0-volume filtration) to remove unreacted reagents. This process is monitored by size-exclusion liquid chromatography (“LC”) at 540 nm and 280 nm. The concentration is adjusted to 4 g/dl Hb and the pH is adjusted to 6.0±7.8 .

The final PEG-Hb conjugate product is sterile filtered using a 0.2-μm sterile disposable capsule and collected into a sterile depyrogenated vessel at 4±2° C. The PEG-Hb conjugate is diluted to 4 g/dl RL and the pH adjusted to 7.4±0.2 pH and then sterile-filtered (0.2 μm) and aliquoted into endotoxin free sterile containers.

### Example 3

### Measurement of Nitrite to Nitric Oxide Reaction

Deoxygenated SFH and PEG-Mal from Example 2 were reacted anaerobically with sodium nitrite in a sealed cuvette in the presence of sodium dithionite. The reaction was monitored spectrophotometrically at various concentrations of excess nitrite. The resulting spectral data were deconvoluted using parent spectra for deoxyhemoglobin, iron-nitrosyl-hemoglobin, and methemoglobin. Since hemoglobin species can deviate from pseudo first-order kinetics for this reaction due to T-to-R state allosteric transition, rate constants were derived from the disappearance of deoxyhemoglobin during the initial phase of the reaction kinetics.

The reaction rates of SFH and PEG-Mal with excess nitrite were linear with nitrite concentration. Analyses of the time courses showed that both reactions had autocatalytic properties. SFH deviated substantially from pseudo first-order kinetics, as expected due to its allosteric transition, while PEG-Mal exhibited only minor cooperativity. SFH and PEG-Mal reduced nitrite to NO with initial rate constants of 0.13 M⁻¹s⁻¹ and 3.6 M⁻¹s⁻¹, respectively, showing a 27-fold higher rate for PEG-Mal compared to SFH.

The examples set forth above are provided to give those of ordinary skill in the art a complete disclosure and description of how to make and use exemplary embodiments of the invention, and are not intended to limit the scope of what the inventors regard as their invention. Modifications of the above-described modes (for carrying out the invention that are obvious to persons of skill in the art) are intended to be within the scope of the following claims. All publications, patents, and patent applications cited in this specification are incorporated herein by reference as if each such publication, patent or patent application were specifically and individually indicated to be incorporated herein by reference.

We claim:

1. A method for delivering oxygen to tissue and reducing nitrite to nitric oxide (NO) in the microvasculature comprising administering a deoxygenated maleimide polyethylene glycol hemoglobin (MalPEG-Hb) conjugate to a subject,
wherein the deoxygenated MalPEG-Hb conjugate has at least 20-fold greater nitrite reductase activity compared to that of stroma free hemoglobin when measured under the same conditions.
